(19) United States
(12) Patent Application Publication
(10) Pub. No.: US 2026/0363995 A1
(43) Pub. Date: Nov. 19, 2026

(54) Dry Shampoo Composition and Method of Use

(71) Applicants: Active Concepts LLC, Lincolnton, NC (US); Beginning is Now, Inc., New York, NY (US)

(72) Inventors: Sanam Fazilova, Charlotte, NC (US); Mark Knitowski, Hillsborough, NJ (US); Julian Moreno, Forest City, NC (US)

(73) Assignees: Active Concepts LLC, Lincolnton, NC (US); Beginning is Now, Inc., Lincolnton, NC (US)

(21) Appl. No.: 19/211,979

(22) Filed: May 19, 2025

(51) Int. Cl. A61K 8/73; A61K 8/02; A61K 8/99; A61Q 5/02; A61Q 5/12

(52) U.S. Cl. CPC A61K 8/732; A61K 8/022; A61K 8/735; A61K 8/99; A61Q 5/02; A61Q 5/12

(57) ABSTRACT

A dry shampoo composition including the following components: (1) cleansing powder ingredient capable of absorbing oils produced by the scalp and/or removing dirt and impurities from the hair, e.g., a starch powder; (2) a conditioning ingredient, e.g., a humectant; and optionally, (3) one or more preservative ingredients. The dry shampoo composition is capable of cleaning the hair and/or scalp, conditioning the hair and/or scalp, and may also reduce sebum production on the scalp. The conditioning ingredient may be a quaternized hyaluronic acid, e.g., hydroxypropyltrimonium hyaluronate. The dry shampoo composition may be a non-aerosol dry shampoo composition that is propellant-free. A method for cleansing and conditioning hair may comprise a step of applying the dry shampoo composition to dry hair to clean and condition the dry hair. The application may be non-aerosol and propellant-free application.

### TECHNICAL FIELD

The present disclosure relates to a 2-in-1 dry shampoo composition that effectively cleanses and conditions the hair while also reducing sebum production on the scalp. The dry shampoo composition can comprise a cleansing component, a conditioning component, and optionally, one or more preservatives. The dry-shampoo can be non-aerosol, propellant-free and/or pigment-free.

### BACKGROUND

Cleansing the hair and scalp with conventional shampoo is a widely practiced grooming routine, essential for maintaining optimal hair cleanliness and health. Hair cleansers are typically used to remove sweat, excess sebum, and impurities on the hair and scalp. Regular hair cleansing also assists with maintaining scalp health by removing environmental pollutants and bacteria from the scalp while reducing the risk of scalp infections. Conventional liquid or gel hair cleansers require water so that the shampoo composition can be rinsed from the hair and are generally followed up with a separate hair conditioning application. This two-step application can be time consuming and can only be accomplished when water is readily available. Dry shampoo, however, is a convenient and time efficient product used to maintain hair cleanliness and health when time is limited, or water is unavailable.

Dry shampoo compositions are a well-known method for cleansing hair. Conventional dry shampoo compositions generally comprises a starch component and requires the use of pigments to mask the noticeable and unappealing residue deposited on to the hair. Dry shampoo compositions on the market also commonly require a propellant to dispense the composition onto the hair. Aerosol propellants are generally used in dry shampoo compositions and more specifically the use of drying alcohols and silicones. Aerosol propellants can strip the hair of moisture, irritate the scalp, and deposit further residue onto the scalp, and can lead to hair buildup and breakage. Furthermore, dry shampoos on the market typically contain several additional filler ingredients such as synthetic components that can further dry out the hair follicles and can cause hair brittleness with frequent usage.

Hyaluronic acid is naturally occurring polysaccharide and is known for its water retention applications in skin and hair care. Hyaluronic acid is a negatively charged substance due to the presence of carboxylate groups on the glucuronic acid residues within the structure. Hyaluronic acid can be chemically modified by quaternization to introduce quaternary ammonium groups to the structure, creating quaternized hyaluronic acid. The addition of quaternary ammonium groups into the structure allows for quaternized hyaluronic acid to become a positively charged molecule. Hydroxypropyltrimonium hyaluronate is a form of quaternized hyaluronic acid and boasts remarkable potential in hair care formulations due to its unique properties. It exhibits enhanced affinity for hair strands, allowing it to adhere effectively to the hair's surface. These characteristics form the foundation for its utilization as a conditioning agent. When incorporated into hair care products, such as conditioners or hair masks, hydroxypropyltrimonium hyaluronate acts as a film-forming agent, creating a protective barrier on the hair's surface. This barrier helps to seal in moisture, preventing dehydration and minimizing damage caused by external aggressors like UV radiation and pollution.

Moreover, hydroxypropyltrimonium hyaluronate possesses excellent moisture-retention capabilities, which further contributes to its effectiveness in hair conditioning. Its molecular structure enables it to attract and bind water molecules, facilitating hydration within the hair shaft. This hydration not only imparts a silky-smooth texture to the hair but also enhances its elasticity and suppleness. By maintaining optimal moisture levels within the hair, hydroxypropyltrimonium hyaluronate helps to prevent issues like brittleness and breakage, promoting overall hair health and vitality.

Furthermore, the positively charged quaternary ammonium groups in hydroxypropyltrimonium hyaluronate interact with the negatively charged surface of the hair cuticle. This interaction promotes smoother hair texture by neutralizing static electricity and reducing friction between individual hair fibers. As a result, combing and styling become easier, and the risk of hair damage from mechanical stress is minimized. Additionally, the conditioning properties of hydroxypropyltrimonium hyaluronate can help to improve the manageability of hair, making it more responsive to styling treatments and reducing the occurrence of tangles and knots. While hydroxypropyltrimonium hyaluronate is generally effective when formulated in solution and in liquid-based cosmetic products, it is uncommonly used in powder based cosmetic products due to its water retention and moisture attracting properties that cause clumping and do not allow the powder to remain dry and free flowing.

Dry shampoo compositions are traditionally designed to absorb sebum on the hair, but they do not reduce sebum production on the scalp. Therefore, there is a need for a dry shampoo composition capable of simultaneously cleansing and conditioning while effectively reducing sebum production on the scalp.

### SUMMARY

In one aspect, a dry shampoo composition is disclosed. The dry shampoo composition may comprise the following components: (1) cleansing powder ingredient and (2) a humectant. Additionally, the dry shampoo composition may optionally include a preservative. The dry shampoo composition may be a non-aerosol dry shampoo composition that is propellant-free.

The dry shampoo composition may comprise a cleansing powder ingredient in an amount of from 55 to 99 wt. %, or in an amount of from 65 to 97 wt. %. The cleansing powder ingredient may be a starch powder. The starch powder may comprise one or more of a rice starch powder, a corn starch powder, tapioca starch powder, a potato starch powder, an aluminum starch powder, or a wheaten starch powder. The starch powder may be a rice starch powder, or a rice starch powder in combination with one or more of a corn starch powder, a tapioca starch a powder, a potato starch powder, an aluminum starch powder, or a wheaten starch powder.

The humectant may be present in the dry shampoo composition in an amount of from 0.1-2.0 wt. %. The humectant may be a modified hyaluronic acid, e.g., a quaternized hyaluronic acid. The quaternized hyaluronic acid may be hydroxypropyltrimonium hyaluronate. The inclusion of hydroxypropyltrimonium hyaluronate into the dry shampoo composition, reduces sebum production on the scalp.

When a preservative is present, the preservative may be present in the dry shampoo composition in an amount of from 0.01 to 2.0 wt. %. The preservative may be Lactobacillus ferment.

In another aspect, a dry shampoo composition is disclosed that comprises the following components: (1) a starch powder in an amount of from 65 wt. % to 97 wt. %; (2) a humectant, wherein the humectant comprises a quaternized hyaluronic acid and is present in an amount of from 0.1 to 2.0 wt. %; and (3) a preservative that is present in an amount of from 0.01 to 2.0 wt. %. The quaternized hyaluronic acid may be hydroxypropyltrimonium hyaluronate. The inclusion of hydroxypropyltrimonium hyaluronate into the dry shampoo composition, reduces sebum production on the scalp.

In some aspects, the dry shampoo may comprise (1) 65-97 wt. % Oryza sativa (Rice) starch powder; (2) 0.1-2.0 wt. % hydroxypropyltrimonium hyaluronate; and (3) 0.01-2.0 wt. % Lactobacillus ferment. Such a composition may reduce sebum production on a scalp when applied thereto. The dry shampoo composition may be a non-aerosol dry shampoo composition that is propellant-free.

In another aspect, a method for cleansing and conditioning hair is disclosed. The method may comprise applying a dry shampoo composition to dry hair in order to clean and condition the dry hair. The dry shampoo composition may comprise: (1) a cleansing powder ingredient; (2) a humectant; and optionally, (3) a preservative. The humectant may comprise quaternized hyaluronic acid. The dry shampoo composition may comprise the following: (1) 65-97 wt. % Oryza sativa (Rice) starch powder; (2) 0.1-2.0 wt. % hydroxypropyltrimonium hyaluronate; and (3) 0.01-2.0 wt. % Lactobacillus ferment. This dry shampoo composition may be applied to dry hair and a scalp, and reduces sebum production on the scalp. Applying the dry shampoo composition may include a non-aerosol and propellant-free application process.

### DESCRIPTION OF THE DRAWINGS

FIG. 1 is a bar graph showing scalp sebum presence over a period of 32 hours for a subject treated with a dry shampoo composition according to the instant disclosure and an untreated control.

FIG. 2 is a line graph showing change in scalp sebum presence over a period of 32 hours for a subject treated with a dry shampoo composition according to the instant disclosure and an untreated control.

FIG. 3 is a bar graph showing scalp hydration over a period of 32 hours for a subject treated with a dry shampoo composition according to the instant disclosure and an untreated control.

FIG. 4 is a line graph showing change in scalp hydration over a period of 32 hours for a subject treated with a dry shampoo composition according to the instant disclosure and an untreated control.

FIG. 5, FIG. 6, and FIG. 7 are images of Sebufix® SF16 Foils showing sebum presence (black areas) on the scalps of five different participants over a period of 32 hours, where half of the participant's scalp is treated with a dry shampoo composition according to the instant disclosure and the other half is left untreated. Scalp skin with lower sebum content shows few small spots, whereas oily skin is displayed by a great number of large spots.

### DETAILED DESCRIPTION

The following detailed description is merely exemplary in nature and is not limited to the disclosure or the application and uses of the subject matter as described herein. The disclosure may be embodied in many different forms and there is no intention to be bound by any theory presented in the preceding background or the following detailed description.

Accordingly, aspects described herein can be understood more readily by reference to the detailed description, examples, figures, etc. Elements, apparatus, and methods described herein, however, are not limited to the specifics presented in the detailed description, examples, and figures. It should be recognized that the exemplary aspects herein are merely illustrative of the principles of the invention. Numerous modifications and adaptations will be readily apparent to those of skill in the art without departing from the spirit and scope of the invention/claims.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

Additionally, in any disclosed aspect, the terms “substantially,” “approximately,” and “about” may be substituted with “within [a percentage] of” what is specified, where the percentage includes 0.1, 1, 5, and 10 percent.

Many different arrangements of the various components and/or steps depicted and described, as well as those not shown, are possible without departing from the scope of the claims below. Aspects of the present technology have been described with the intent to be illustrative rather than restrictive. Alternative aspects will become apparent from reference to this disclosure. Alternative means of implementing the aforementioned can be completed without departing from the scope of the claims below. Certain features and sub-combinations are of utility and can be employed without reference to other features and sub-combinations and are contemplated within the scope of the claims.

### Dry Shampoo Composition

The present disclosure relates to a dry shampoo composition, a 2-in-1 dry shampoo composition, and/or a 2-in-1 non-aerosol and propellant-free dry shampoo composition. In some aspects, the 2-in-1 dry shampoo composition is capable of cleansing and conditioning the hair, while also reducing sebum production on the scalp.

Within the present disclosure, a dry shampoo shall be understood to relate to a product or composition capable of cleansing the hair and/or scalp without needing to be applied to wet hair, without needing to be lathered, and/or without needing to be rinsed from the hair. Typically, dry shampoos utilize alcohol-based and/or starch-based cleansing ingredients to absorb sweat, sebum, and/or oils on the hair. Wet shampoo, conversely, is understood to be a hair cleansing product or composition that is applied to wet hair, lathered, and then rinsed from the hair using water. Wet shampoos typically utilize surfactants that remove oils from the hair when the product is rinsed.

A dry shampoo that includes mainly a cleansing powder ingredient, e.g., a starch powder, is a powder-based dry shampoo. A powder-based dry shampoo comprises more than 50 wt. %, more than 55 wt. %, more than 60 wt. %, more than 65 wt. %, more than 70 wt. %, more than 75 wt. %, more than 80 wt. %, more than 85 wt. %, more than 90 wt. %, or more than 95 wt. % of a cleansing powder ingredient.

The term “non-aerosol” is understood to mean that the dry shampoo does not contain any propellants, including but not limited to liquified gas propellants (e.g., butane, isobutane, propane, dimethyl ether, liquified petroleum gas, etc.), chlorofluorocarbon aerosol can propellants, and compressed gas propellants (e.g., nitrogen, nitrous oxide, carbon dioxide). In other words, the dry shampoo is propellant-free. Propellants such as butane, isobutane, and propane have been identified as sources of benzene identified in aerosol dry shampoos.

Many traditional dry shampoo compositions require pigments or colorants to be added to dry shampoo formulations to hide the presence of residue when used on different hair colors. In some aspects, the dry shampoo composition according to the present disclosure may not contain a colorant or pigment, or any coloring adhesion promotor. The dry shampoo composition can be used on any hair type or texture without leaving behind traces of light-colored residue. Accordingly, there is no need to incorporate a pigment or colorant into the composition.

In some aspects, the dry shampoo composition described herein may not contain silicones (e.g., polyorganosiloxane), silicone oil, silicone gum, and/or any other silica. If silicones are used, water-soluble silicones may be preferred because they are less likely to build up on the hair.

In some aspects, the dry shampoo composition described herein may not include a water-insoluble mineral, a moisturizer such as hemisqualane, calcium carbonate, or a monoalcohol.

The dry shampoo composition described herein may comprise, consist of, or consist essentially of the following components: (1) a cleansing ingredient capable of absorbing oils produced by the scalp and/or removing dirt and impurities from the hair, e.g., a starch powder; (2) a conditioning ingredient, e.g., a humectant; and optionally, (3) one or more preservative ingredients. In addition to (1), (2), and (3), the dry shampoo composition may comprise additional ingredients, including but not limited to synthetic or natural fragrances, colorants, etc.

### (1) a Cleansing Ingredient

The cleansing ingredient is not so limited so long as it is or includes an ingredient capable of absorbing oils produced by the scalp and/or removing dirt and impurities from the hair. For example, the cleansing ingredient is capable of absorbing excess sebum and/or oil present on the hair and/or scalp. Typical cleansing ingredients used in dry shampoos include alcohol-based cleansing ingredients, e.g., ethanol, and starch-based or powder-based cleansing ingredients, e.g., rice starch powder.

In a preferred aspect, the cleansing ingredient of the dry shampoo may comprise, consist of, or consist essentially of a cleansing powder ingredient.

In a preferred aspect, the cleansing ingredient comprises, consists of, or consists essentially of a cleansing powder ingredient. In one aspect, the cleansing powder ingredient may be present in the cleansing ingredient in an amount of about 55 wt. % or more, about 60 wt. % or more, about 65 wt. % or more, about 70 wt. % or more, about 75 wt. % or more, about 80 wt. % or more, about 85 wt. % or more, about 90 wt. % or more, about 95 wt. % or more, about 96 wt. % or more, about 97 wt. % or more, or about 98 wt. % or more, and about 99 wt. % or less, 98 wt. % or less, or 97 wt. % or less. In one preferred aspect, the cleansing powder ingredient may be present in the cleansing ingredient in an amount of from about 55 wt. % to about 99 wt. %, from about 65 wt. % to about 99 wt. %, from about 75 wt. % to about 99 wt. %, from about 85 wt. % to about 99 wt. %, or from about 95 wt. % to about 99 wt. %. In another preferred aspect, the cleansing powder ingredient may be present in the cleansing ingredient in an amount of from about 65 wt. % to about 97 wt. %, from about 75 wt. % to about 97 wt. %, from about 85 wt. % to about 97 wt. %, or from about 95 wt. % to about 97 wt. %.

The cleansing powder ingredient is not so limited, and may comprise, consist of, or consist essentially of one or more of silica, clay (e.g., kaolin clay or bentonite clay) cocoa powder, starch powder (e.g., arrowroot powder, tapioca starch powder, corn starch powder, rice starch powder, potato starch powder, wheaten starch powder, aluminum starch, etc.), or talc.

In one preferred aspect, the cleansing powder ingredient may comprise, consist of, or consist essentially of a starch powder. For example, the starch powder may comprise, consist of, or consist essentially of one or more starch powders selected from an arrowroot powder, a tapioca starch powder, a corn starch powder, a rice starch powder, a potato starch powder, a wheaten starch powder, or an aluminum starch powder. In another preferred aspect the starch powder may comprise, consist of, or consist essentially of a rice starch powder or a rice starch powder in combination with one or more of an arrowroot powder, a tapioca starch powder, a corn starch powder, a potato starch powder, a wheaten starch powder, and an aluminum starch powder.

In some aspects, the cleansing powder ingredient may comprise, consist of, or consist essentially of Oryza sativa (rice) starch powder. The weight percentage of Oryza sativa (rice) starch powder in the dry shampoo composition, based on the total weight of the dry shampoo composition, can be from about 65 wt. % to about 97 wt. %, from about 75 wt. % to about 97 wt. %, from about 85 wt. % to about 97 wt. %, or from about 95 wt. % to about 97 wt. %.

### (2) a Conditioning Ingredient

The conditioning ingredient is not so limited, and can be any ingredient that moisturizes the hair. The conditioning ingredient may also provide one or more of shine, softness, smoothness, /or strength to the hair. Hair conditioning applications hydrate the hair and/or scalp and may provide protection against hair breakage.

In one aspect, the conditioning ingredient may be present in an amount of from about 0.01 wt. % to about 10 wt. %, from about 0.1 wt. % to about 9 wt. %, from about 0.1 wt. % to about 8 wt. %, from about 0.1 wt. % to about 7 wt. %, from about 0.1 wt. % to about 6 wt. %, from about 0.1 wt. % to about 5 wt. %, from about 0.1 wt. % to about 4 wt. %, from about 0.1 wt. % to about 3 wt. %, from about 0.1 wt. % to about 2 wt. %, from about 0.1 wt. % to about 1.5 wt. %, or from about 0.1 wt. % to about 1 wt. %. In one preferred aspect, the conditioning ingredient may be present in an amount of from about 0.1 wt. % to about 2 wt. %, from about 0.1 wt. % to about 1.5 wt. %, from about 0.1 wt. % to about 1 wt. %, or from about 0.1 wt. % to about 0.5 wt. %.

In some aspects, the conditioning ingredient may be or include a hydrophilic conditioning ingredient capable of hydrating and moisturizing the hair and/or scalp. Examples of hydrophilic conditioning ingredients include, but are not limited to, glycerin, sorbitol, sodium lactate, panthenol, aloe vera, hyaluronic acid, amino acids (e.g., arginine and lysine) betaines, etc.

In some aspects, the conditioning ingredient may be or include a humectant. Humectants are hydrophilic ingredients that draw moisture in from the surrounding environment, i.e., they are hygroscopic. Examples of humectants include, but are not limited to, urea, lactic acid, hyaluronic acid, and glycerin.

In some preferred aspects, the humectant may be or include a modified form of hyaluronic acid that remains hydrophilic. For example, a hyaluronic acid that has been modified to include positively charged functional groups may be used. An example of such a modified form of hyaluronic acid is quaternized hyaluronic acid. Quaternization is a chemical reaction that involves a nitrogen atom and the addition of a quaternary ammonium group to the structure. For example, one or more amine groups of a hyaluronic acid may be converted to a quaternary ammonium group. An exemplary quaternized hyaluronic acid that may be used is hydroxypropyltrimonium hyaluronate.

In some aspects, the hydroxypropyltrimonium hyaluronate contains positively charged quaternary ammonium groups that interacts with negatively charged hair strands which allows it to adhere effectively to the hair's surface. Furthermore, the enhanced cationic charge of hydroxypropyltrimonium hyaluronate enables it to attract and bind to water molecules, facilitating hydration withing the hair shaft. In some embodiments, the hydroxypropyltrimonium also acts as a film-forming agent, creating a protective barrier on the hair's surface. This barrier helps to seal in moisture, prevents dehydration, and minimizes damage caused by external aggressors such as UV and pollutants.

The inclusion of hydroxypropyltrimonium hyaluronate in the 2-in-1 dry shampoo composition unexpectedly reduces sebum production on the scalp. Traditional dry shampoos are known to absorb sebum on the scalp by using, for example, a starch powder, but they do not reduce sebum production. Hydroxypropyltrimonium hyaluronate, however, interacts with hair fibers and scalp pores to greatly reduce the sebum production on the scalp. While quaternized hyaluronic acid is known for its conditioning capabilities, it is novel and unexpected that hydroxypropyltrimonium hyaluronate is capable of reducing sebum production on the scalp.

In some embodiments of the present invention, the weight percentage of hydroxypropyltrimonium hyaluronate in the dry shampoo composition, based on the total weight of the dry shampoo composition, can be about 0.1 wt. % to about 2 wt. %, from about 0.1 wt. % to about 1.5 wt. %, from about 0.1 wt. % to about 1 wt. %, or from about 0.1 wt. % to about 0.5 wt. %.

### (3) Preservative Ingredient

The preservative ingredient is an optional ingredient of the dry shampoo composition according to the present disclosure. However, in some aspects, the dry shampoo composition according to the present disclosure may comprise at least one preservative. In some aspects, the preservative may be at least one selected from an antimicrobial, an antifungal, or a combination thereof. These preservative ingredients may be added to extend shelf life.

In some aspects, the preservative system may be Lactobacillus ferment Saccharomyces ferment, or a combination thereof. Lactobacillus ferment is created by fermenting Lactobacillus bacteria in a defined growth media and it acts as a broad-spectrum antimicrobial.

In some aspects, the preservative may be present in the dry shampoo composition in the amount of from about 0.01 wt. % to about 2 wt. %, from about 0.01 wt. % to about 1.5 wt. %, from about 0.01 wt. % to about 1 wt. %, from about 0.01 wt. % to about 0.5 wt. %, from about 0.01 wt. % to about 0.25 wt. %, from about 0.01 wt. % to about 0.1 wt. %, and preferably in the amount of about 2 wt. %.

### Method of Using Dry Shampoo Composition

A method of using the dry shampoo composition described herein is not so limited. In some aspects, the dry shampoo composition may be used to clean the hair, e.g., through the oil absorbing action of the cleansing ingredient. In other aspects, the dry shampoo composition may be used both to clean the hair, e.g., through the oil absorbing action of the cleansing ingredient, and to condition the hair, e.g., through the action of the conditioning ingredient. In other aspects, the dry shampoo composition may be used to clean the hair, condition the hair, and to reduce sebum production on the scalp. Without wishing to be bound by any particular theory, the ability to reduce sebum production on the scalp is believed to be a result of the addition of a quaternized hyaluronic acid, e.g., hydroxypropyltrimonium hyaluronate.

The method of using the dry shampoo composition described herein to clean the hair, to condition the hair, and/or to reduce sebum production on the scalp may comprise a step of applying a dry shampoo composition to the hair and/or scalp. The dry shampoo composition applied to the hair and/or scalp may do the following: (1) clean the hair and/or scalp; (2) clean and condition the hair and/or scalp; or (3) clean and condition the hair and/or scalp, and reduce sebum production on the scalp. In a preferred aspect, at the time of application, the hair and scalp are dry.

The present disclosure provides a 2-in-1 dry shampoo composition with, at least, both cleaning and conditioning capabilities. The present invention allows for the hair and scalp to be cleaned and conditioned simultaneously without the need for separate application of a separate conditioning composition. In some aspects, the 2-in-1 dry shampoo composition may also act to reduce sebum production on the scalp.

In some embodiments, the dry shampoo composition can be topically applied to the hair and massaged into the scalp to activate ingredients until powder is absorbed onto the scalp. The applied dry shampoo composition can be massaged into the hair and scalp to cleanse the hair of dirt and impurities and reduce sebum production on the scalp. The dry shampoo composition can be optionally brushed through the hair using a brush or a comb or distributed through the hair with the user's hands. In some embodiments, the dry shampoo composition can be applied topically to the hair and scalp directly using a powder dispensing bottle.

In a preferred aspect, the application of the dry shampoo composition is a non-aerosol application. This means that the dry shampoo composition is applied without the use of propellants or any expanding materials for employing the composition from a container.

### EXAMPLES

Hereinafter, the present application is specifically described by way of examples, but the scope of the present application is not limited by the following examples.

A sebum reduction study was conducted to evaluate the ability of 2-in-1 dry shampoo and conditioner to decrease sebum production on the scalp while maintaining moisturization. The 2-in-1 dry shampoo composition was prepared according to the present disclosure, the formula of which is provided in Table 1. The ingredients in Table 1 were combined to form the 2-in-1 dry shampoo composition.

TABLE 1
2-in-1 Dry Shampoo Composition

| Material | Weight Percentage Range |
|---|---|
| Oryza sativa (Rice) starch powder | 65-97% |
| Hydroxypropyltrimonium hyaluronate | 0.1-2.0% |
| Lactobacillus ferment | 0.01-2.0% |

The materials used in the testing included the equipment Dermalab Skin Combo (Hydration Probe and DermaScope Camera Probe) and Sebufix® SF16. The software used included ImageJ (National Institutes of Health (NIH)) and Excel® Analysis ToolPak.

Participants between the ages of 23 and 55, with unwashed second day hair were selected to participate in the study. Each participant separated their hair down the middle and had both halves of their scalp randomly assigned to a specific condition displayed in Table 2. Baseline moisturization and sebum measurements were taken on the middle of the scalp prior to treatment. Following baseline measurements, half of the participant's scalp was treated with 2-in-1 dry shampoo and conditioner, while the other half of their scalp was left untreated. Treatment involved applying the 2-in-1 dry shampoo to the participant's scalp and base of hair, and massaging the 2-in-1 dry shampoo into the hair and scalp manually. Application of the test article was applied once during the 32-hour test period. Participants were instructed not to wash their hair 48 hours prior to baseline measurements. After application, participants were instructed not to use any other hair care products throughout the duration of the study. Sebum and moisturization measurement were recorded after the application of the test article.

TABLE 2
Descriptions of the Treatments for Each Test Site

| Scalp Test Site | Treatment/Test Article Application Description |
|---|---|
| 1 | Untreated Control |
| 2 | 2-In-1 Dry Shampoo |

Sebufix® SF16 foils were used to measure scalp content. The Sebufix® SF16 foils absorb sebum present on the skin's surface and display spots in different sizes representative of the sebum content. Sebum present on the foils appears as black areas on the images. The more area covered in black, the more sebum content present. Sebum measurements were taken at baseline, 1 hour, 8 hours, 24 hours, and 32 hours after product application on the middle of the scalp. Images of Sebufix® SF16 foils were taken and analyzed in ImageJ analysis software (NIH) to quantify the amount of sebum present on the scalp.

An average of three consecutive sebum and hydration measurements per condition at each time point was recorded and expressed as area of sebum (Pixel²) and micro-Siemens (μS), respectively, for each volunteer. Data were displayed as averages from all volunteers and analyzed using t-tests with statistical significance accepted at p≤0.05. Percent change was calculated for each condition at every timepoint relative to Baseline values.

The data obtained for this study met criteria for a valid study as the Untreated Control test site performed as expected. Application of 2-in-1 Dry Shampoo once in a 32-hour period demonstrated effective immediate and short-term sebum reducing properties while also enhancing scalp hydration throughout the study duration.

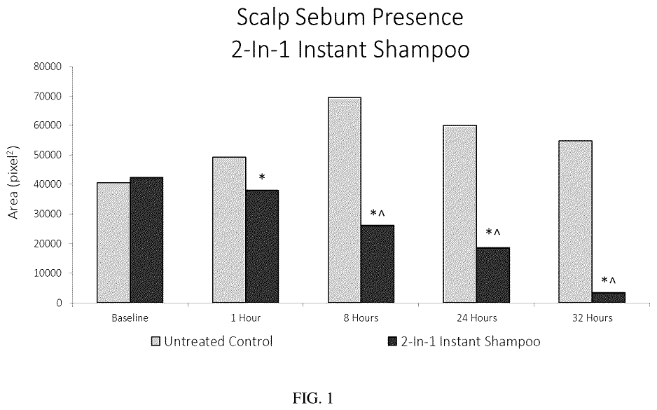

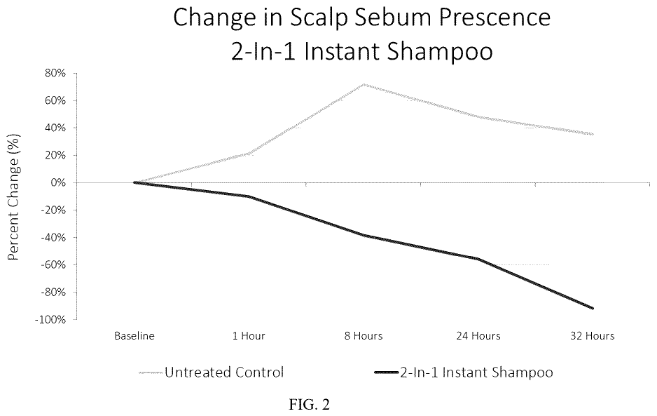

This study was conducted to evaluate the sebum production reducing properties of 2-In-1 Dry Shampoo on the scalp, while maintaining moisturization. When compared to baseline values, the Untreated Control increased scalp sebum content eight hours and 32 hours after baseline measurements by 72% and 35%, respectively FIG. 1, FIG. 2; Table 3). Conversely, 2-In-1 dry Shampoo significantly reduced sebum levels on the scalp by 38% and 92% eight hours and 32 hours after application, compared to baseline measurements, respectively (FIG. 1, FIG. 2; Table 3). These results indicate 2-In-1 Dry Shampoo effectively suppresses excess sebum levels present on the scalp up to 32 hours after one application.

TABLE 3

| Results from T-test Analysis of Sebum Presence from | | |
|---|---|---|
| Baseline to 1 Hour, 8 Hours, 24 | | |
| Hours, and 32 Hours After Application. The asterisk | | |
| indicates significance (p ≤ 0.05) compared | | |
| to Baseline values. | | |
| | Untreated Control | 2-In-1 Dry Shampoo |
| 1 Hour | 0.714 | 0.026* |
| After Application | | |
| 8 Hours | 0.208 | 0.010* |
| After Application | | |
| 24 Hours | 0.240 | 0.005* |
| After Application | | |
| 32 Hours | 0.350 | 0.032* |
| After Application | | |

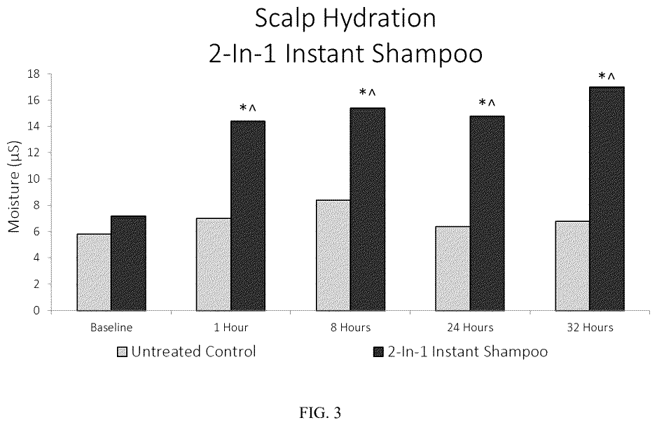

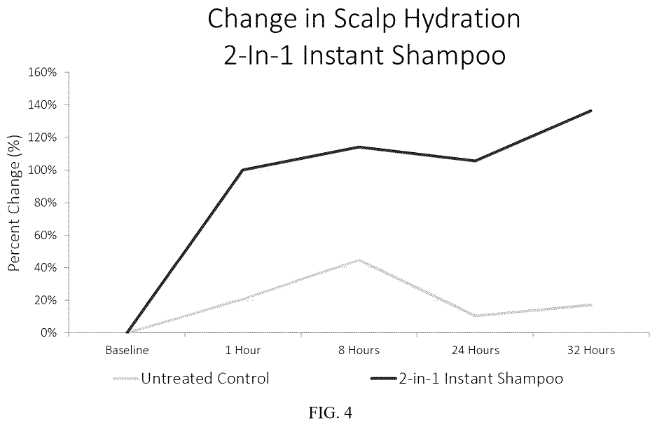

The Untreated Control exhibited an increase in scalp hydration by 45% and 17% eight hours and 32 hours after application, respectively (FIG. 3 and FIG. 4; Table 4). However, 2-In-1 Instant Shampoo significantly increased hydration levels on the scalp by 114% and 136% eight hours and 32 hours after application, respectively (FIG. 3 and FIG. 4; Table 4). These results demonstrate 2-In-1 Dry Shampoo enhances scalp hydration up to 32 hours after one application.

TABLE 4

| Results from T-test Analysis of Moisture Levels | | |
|---|---|---|
| from Baseline to After 1 Hour, 8 Hours, 24 Hours, | | |
| and 32 Hours After Application. The | | |
| asterisk indicates significance (p ≤ 0.05) | | |
| compared to Baseline values. | | |
| | Untreated | 2-In-1 Instant |
| | Control | Shampoo |
| 1 Hour | 0.557 | 0.047* |
| After Application | | |
| 8 Hours | 0.177 | 0.029* |
| After Application | | |
| 24 Hours | 0.089 | 0.082* |
| After Application | | |
| 32 Hours | 0.093 | 0.012* |
| After Application | | |

Similar results are shown when examining the collective effect between each condition for both sebum presence and hydration on the scalp. Applying 2-In-1 Dry Shampoo significantly reduced scalp sebum content while also augmenting hydration up to 32 hours after application (FIG. 1, FIG. 2, FIG. 3, and FIG. 4; Table 5, Table 6). These results indicate 2-In-1 Dry Shampoo effectively reduces excess sebum present on the scalp while augmenting hydration to prevent scalp irritation after application.

TABLE 5
Results from T-test Analysis of Sebum Presence After 1 Hour, 8 Hours, 24 Hours, and 32 Hours
After Application. ? indicates significance (p ≤ 0.05) between conditions within the same timepoint.

| | 1 Hour After | 8 Hours After | 24 Hours After | 32 Hours After |
|---|---|---|---|---|
| | Application: | Application: | Application: | Application: |
| | Untreated Control vs | Untreated Control vs | Untreated Control vs | Untreated Control vs |
| | 2-In-1 Dry Shampoo | 2-In-1 Dry Shampoo | 2-In-1 Dry Shampoo | 2-In-1 Dry Shampoo |
| P-value | 0.140 | 0.017? | 0.004? | 0.013? |

TABLE 6
T-test Analysis of Moisture Levels After 1 Hour, 8 Hours, 24 Hours, and 32 Hours After Application.
? indicates significance (p ≤ 0.05) compared to Base Shampoo and Base Conditioner
within the same timepoint.

| | 1 Hour After | 8 Hours After | 24 Hours After | 32 Hours After |
|---|---|---|---|---|
| | Application: | Application: | Application: | Application: |
| | Untreated Control vs | Untreated Control vs | Untreated Control vs | Untreated Control vs |
| | 2-In-1 Instant | 2-In-1 Instant | 2-In-1 Instant | 2-In-1 Instant |
| | Shampoo | Shampoo | Shampoo | Shampoo |
| P-value | 0.038? | 0.042? | 0.047? | 0.002? |

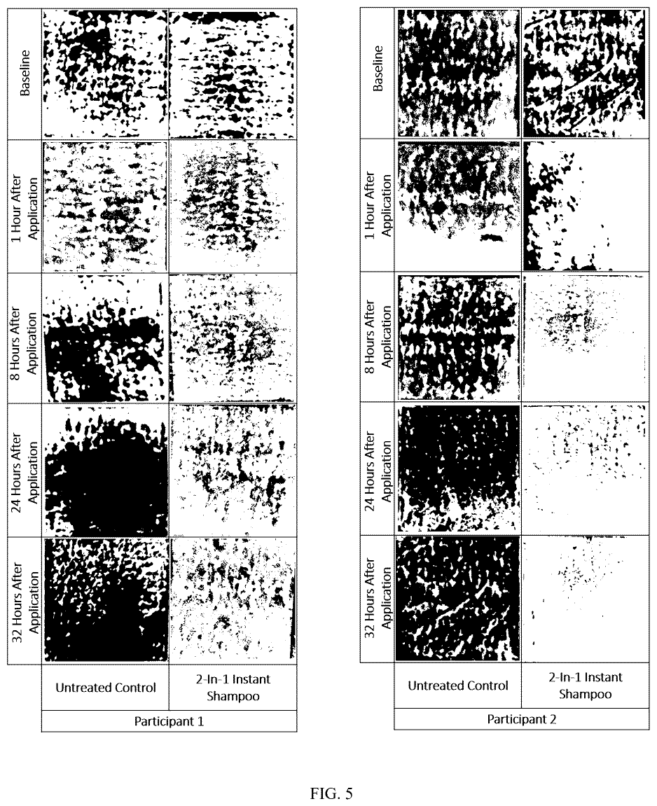

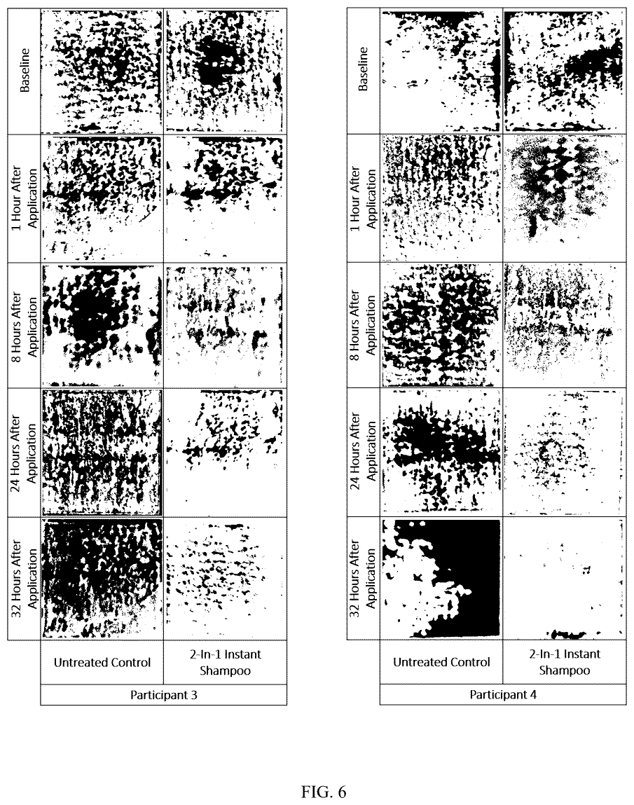

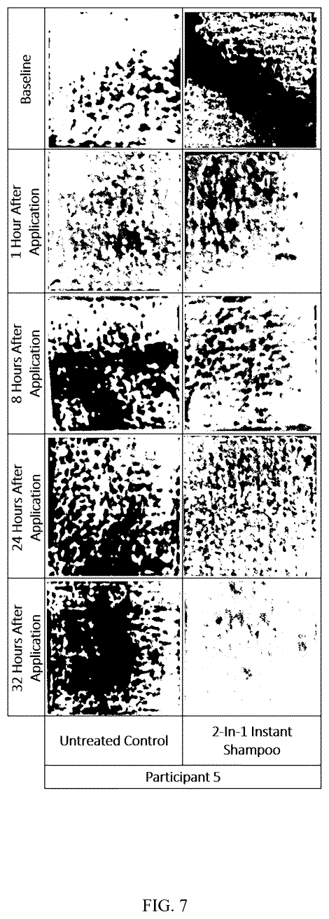

Visually, the half of each scalp treated with 2-In-1 Dry Shampoo experienced a decrease in sebum, while the untreated half demonstrated an increase in scalp sebum (FIG. 5, FIG. 6, FIG. 7). These results demonstrate 2-In-1 Dry Shampoo elicits a visual reduction in scalp sebum with one application.

Taken together, the present study indicates 2-In-1 Dry Shampoo reduces sebum production on the scalp and increases scalp hydration levels for up to 32 hours after one application. Collectively, 2-In-1 Dry Shampoo suppresses sebum and mitigates the shiny and greasy appearance from an oily scalp, while simultaneously enhancing scalp moisturization to prevent the negative effects of dry, red, and itchy skin on the scalp typically associated with an insufficient amount of sebum.

1. A dry shampoo composition, comprising:
a cleansing powder ingredient;
a humectant; and optionally,
a preservative.

2. The dry shampoo composition of claim 1, wherein the dry shampoo composition is a non-aerosol dry shampoo composition that is propellant-free.

3. The dry shampoo composition of claim 1, comprising the cleansing powder ingredient in an amount of from 55 to 99 wt. %.

4. The dry shampoo composition of claim 1, comprising the cleansing powder ingredient in an amount of from 65 to 97 wt. %.

5. The dry shampoo composition of claim 1, wherein the cleansing powder ingredient is a starch powder.

6. The dry shampoo composition of claim 5, wherein the starch powder comprises one or more of rice starch powder, corn starch powder, tapioca starch powder, a potato starch powder, aluminum starch powder, or wheaten starch powder.

7. The dry shampoo composition of claim 6, wherein the starch powder is rice starch powder or rice starch powder in combination with one or more of corn starch powder, tapioca starch powder, a potato starch powder, aluminum starch powder, or wheaten starch powder.

8. The dry shampoo composition of claim 1, wherein the humectant is present in an amount of from 0.1-2.0 wt. %.

9. The dry shampoo composition of claim 1, wherein the humectant is a quaternized hyaluronic acid.

10. The dry shampoo composition of claim 9, wherein the quaternized hyaluronic acid is hydroxypropyltrimonium hyaluronate, and due to the inclusion of hydroxypropyltrimonium hyaluronate, the dry shampoo composition reduces sebum production on the scalp.

11. The dry shampoo composition of claim 1, comprising a preservative that is present in an amount of from 0.01 to 2.0 wt. %.

12. The dry shampoo composition of claim 11, wherein the preservative is Lactobacillus ferment.

13. A dry shampoo composition, comprising:
a starch powder in an amount of from 65 to 97 wt. %;
a humectant, wherein the humectant comprises a quaternized hyaluronic acid and is present in an amount of from 0.1 to 2.0 wt. %; and optionally,
a preservative that is present in an amount of from 0.01 to 2.0 wt. %.

14. The dry shampoo of claim 13, wherein the quaternized hyaluronic acid is hydroxypropyltrimonium hyaluronate, and due to the inclusion of hydroxypropyltrimonium hyaluronate, the dry shampoo composition reduces sebum production on the scalp.

15. The dry shampoo of claim 13, comprising
65-97 wt. % Oryza sativa (Rice) starch powder;
0.1-2.0 wt. % hydroxypropyltrimonium hyaluronate; and
0.01-2.0 wt. % Lactobacillus ferment, wherein
when the dry shampoo composition is applied to a scalp, it reduces sebum production on the scalp.

16. The dry shampoo composition of claim 13, wherein the dry shampoo composition is a non-aerosol dry shampoo composition that is propellant-free.

17. A method for cleansing and conditioning hair, comprising:
applying a dry shampoo composition to dry hair in order to clean and condition the dry hair, wherein the dry shampoo composition comprises:
a cleansing powder ingredient;
a humectant; and optionally,
a preservative.

18. The method of claim 17, wherein the humectant comprises a quaternized hyaluronic acid.

19. The method of claim 17, wherein the dry shampoo composition comprises:
65-97 wt. % Oryza sativa (Rice) starch powder;
0.1-2.0 wt. % hydroxypropyltrimonium hyaluronate; and
0.01-2.0 wt. % Lactobacillus ferment, and wherein
the dry shampoo composition is also applied to a scalp and reduces sebum production on the scalp.

20. The method of claim 17, wherein applying the dry shampoo composition includes a non-aerosol and propellant-free application process.
